(19) United States
(12) Patent Application Publication
(10) Pub. No.: US 2023/0190520 A1
(43) Pub. Date: Jun. 22, 2023

(54) MEDICAL BATTERY MODULE, VACUUM MATTRESS, HEATING MAT

(71) Applicant: Andreas KÖRNER, Naundorf (DE)

(72) Inventor: Andreas KÖRNER, Naundorf (DE)

(21) Appl. No.: 17/924,930

(22) PCT Filed: Jan. 12, 2021
(86) PCT No.: PCT/EP2021/050490

§ 371 Date: Nov. 11, 2022

(30) Foreign Application Priority Data
May 13, 2020 (EP) 202020102683.6

(51) Int. Cl. A61F 7/08; A61F 7/00; A61G 7/057

(52) U.S. Cl. CPC A61F 7/08; A61F 7/007; A61G 7/05753; A61F 2007/008

(57) ABSTRACT

A medical battery module for vacuum mattresses and heating mats includes a battery compartment and a battery that is removable from and insertable into the battery compartment. The battery is electrically connectable or connected to a heating support of the vacuum mattress or of the heating mat. The battery compartment is padded with a shock-resistant material and the shock-resistant material is at least partly sheathed by a liquid-repellent film. The battery module is either separate from the heating support or lies on the heating support.

### CROSS-REFERENCE TO RELATED APPLICATION

This application is a U.S. National Stage Application of International Application No. PCT/EP2021/050490, tiled Jan. 12, 2021, which claims priority to German Application No. 202020102683.6, filed May 13, 2020, the contents of each of which are hereby incorporated by reference.

### BACKGROUND

### Technical Field

The present disclosure relates to a medical battery module for vacuum mattresses and heating mats, the medical battery module comprising a battery compartment and a battery that is removable from and insertable into the battery compartment, wherein the battery is electrically connectable or connected to a heating support, wherein the battery, compartment is padded with a shock-resistant material and the shock-resistant material is at least partly sheathed by a liquid-repellent film. The disclosure further relates to a vacuum mattress or a heating mat comprising such a medical battery module.

### Background Information

In medical emergencies, it is generally necessary to treat a patient quickly and efficiently. Various types of vehicles are used to transport patients to a hospital. In this respect, stretchers, vacuum mattresses and the like are used in the vehicles. The requirements for such an apparatus are set out in EN 1865 and EN 1789, respectively, that inter alia specify common minimum requirements for the testing, equipping for the transport, construction of stretchers and other patient transport means that are used in ambulances or rescue vehicles for the treatment and transport of patients.

### SUMMARY

The currently most common apparatus is usually only suitable for the use within vehicles or buildings and usually not for a. longer use outside vehicles and buildings,

A temperature-regulating apparatus can be used to provide an apparatus for mobile use outside vehicles. In emergency situations, it has been shown that patients can be helped by supplying cold or heat since e.g. the blood vessels can dilate due to the supply of heat. The blood flow is thereby stimulated and the muscles can relax. Due to the supply of heat, patients can also be helped after a medical emergency, for example an accident or an acute illness, through the supply of heat.

In contrast to this, the blood vessels contract on the supply of cold and the muscles can tense up. The exiting of blood, e.g. into surrounding tissue, can be prevented in this respect. By regulating the temperature to different temperatures, a patient can thus be treated in a more targeted manner outside medical facilities or inside or outside vehicles even in the case of very serious injuries.

A disadvantage of such temperature-regulating stretcher supports is that they are often cabled within the vehicle and can thus form a hazard zone in the vehicle. Due to the cabling of such temperature-regulating apparatus, the service life of such apparatus can also be impaired.

It is an object of the present disclosure to provide an improved power supply for a temperature-regulating apparatus that has a longer life and that can be used without a cabling effort. It is a further object of the present disclosure to provide an apparatus that makes it possible to minimize a space requirement within a vehicle.

This object is satisfied by a medical battery module having the features disclosed herein.

Such a medical battery module for vacuum mattresses and heating mats comprises a battery compartment and a battery that is removable from and insertable into the battery compartment, wherein the battery is electrically connectable or connected to a heating support, wherein the battery compartment is padded with a shock-resistant material and the shock-resistant material is at least partly sheathed by a liquid-repellent film, and wherein the battery module is either separate from the heating support or lies on the heating support.

Such a battery module can be easily used with both a vacuum mattress and a heating mat Depending on the application, the battery module can be fixedly or releasably coupled to the vacuum mattress or the heating mat so that the use of such an apparatus can be adapted to the given situation.

The battery compartment can be manufactured from the shock-resistant material and a battery housing can then be arranged in the battery compartment to remove a battery from or to insert a battery into the housing, wherein the battery and the battery housing can then be protected against shocks by the battery compartment.

The battery compartment can comprise two half shells. Such battery compartments can be manufactured particularly simply.

The shock-resistant material or the battery compartment can also consist of two or more components.

A control unit of the battery and of the heating support can be arranged at one side of the medical battery module. In this respect, the use of the battery module can be made particularly simple.

The shock-resistant material can comprise a material selected from the group comprising plastics, thermoplastics, foam comprising isocyanates and polyols, polyurethane, TPU, styrofoam, and the combinations thereof.

The liquid-repellent film or the liquid-repellent layer can comprise a material from the group of thermoplastic elastomers, such as thermoplastic polyurethane (TPU), foam, polyurethane, styrofoam, polystyrene, and further substances and their combinations thereof.

The material of the battery compartment can comprise a plastic, in particular a thermoplastic. Such materials can be used in a relatively simple manufacturing process for the battery compartment or for components of the battery housing.

The thermoplastic can be a material from the group of thermoplastics comprising: acrylonitrile butadiene styrene (ABS), polyamides (PA), polylactide (PLA), polymethyl methacrylate (PMMA), polycarbonate (PC), polyethylene terephthalate (PET), polyethylene (PE), polypropylene (PP), polystyrene (PS), polyether ether ketone (PEEK), poly vinyl chloride (PVC), and combinations thereof. Such materials can be used in an injection molding process to manufacture the battery compartment, the battery housing, or components thereof in a simple, reproducible, and cost-effective manner.

A base of the medical battery module can be arranged, in particular directly, on the heating mat or the vacuum mattress. Space in the vehicle can be saved in this respect.

The medical battery module can be completely sheathed by the liquid-repellent film except for an opening at the front side or the base of the battery compartment. The battery is hereby protected against liquids.

The battery compartment preferably comprises a battery housing and the battery can be removed from the battery housing and can be repeatedly inserted into the battery housing. An electrical connection between the battery and the electronic components of the vacuum mattress or the heating mat can be improved by such a battery housing and protection against an entry of a liquid into the vacuum mattress or the heating mat can also be improved by such a battery housing.

The battery housing preferably comprises a multi-part housing that is at least liquid-repellent, preferably liquid-tight. Since the battery housing can be configured as liquid-repellent, an entry of liquid into the battery housing can be avoided so that the risk of a short circuit in one or more electronic components, which can be arranged in the vacuum mattress, can be minimized.

The heating support particularly preferably has a temperature-regulating film, wherein the temperature-regulating film can be regulated to an at least substantially constant temperature in the range from 15 to 42° C., preferably from 16 to 30° C., in particular from 20 to 30° C., specifically from 22 to 26° C., wherein the temperature can be set in dependence on the patient by a control unit. These temperature ranges are perceived as pleasant by patients who are transported on the vacuum mattress and enable a heating or a cooling of the body of the patient.

The battery housing preferably has an upper shell, a lower shell, and a cover, wherein the upper shell is connected to the lower shell and the cover is releasably coupled to the upper shell and to the lower shell, in particular by a releasable snap-in connection. Such a battery housing can be easily manufactured, enables a simple insertion and removal of the battery, and furthermore provides a desired protection against external impairments such as liquid and impacts.

The battery is preferably connected to the cover and the battery can be removed from the battery housing and can be inserted into the battery housing together with the cover. The battery can be more easily accommodated in the battery housing by such a cover.

The cover preferably has a frame in which the battery is arranged. The battery can be repeatedly and more precisely inserted into the battery housing by such a frame.

A seal is preferably arranged between the cover and the upper shell as well as the lower shell. Since the cover is usually arranged at the outer side of the support, the seal usually likewise seals from the outside such that no liquid, such as water, detergent, disinfectant, blood or the like, can enter the vacuum mattress.

The battery housing and/or the cover is/are preferably manufactured from a plastic, preferably in an injection molding process. The components of the battery housing can hereby be manufactured in a cost-effective and reproducible manner.

The seal is preferably arranged between the cover and a groove of an outer frame, wherein the outer frame connects the upper shell to the lower shell. The connection between the housing parts of the battery housing can be improved by such an outer frame.

The control unit is preferably arranged in the region of the connection between the upper shell and the lower shell, preferably arranged at the outer frame. The control unit can hereby be particularly securely attached to the battery housing.

The battery housing preferably further has a reception space for at least one board on which at least one electronic circuit is provided to communicate with the battery. Such an electronic circuit can thus be arranged more securely in the vacuum mattress than without such a battery housing.

In accordance with a further aspect of the disclosure, the disclosure relates to a vacuum mattress comprising a medical battery module, wherein the medical battery module is welded or adhesively bonded to the vacuum mattress and is electrically connected to a heating support of the vacuum mattress. With a heat-regulating vacuum mattress, patients can, for example, in an emergency situation be supported by the addition of heat.

The heating support can be arranged at an upper side of the vacuum mattress. It has namely been found that the vacuum mattress as such is relatively sensitive to maintain its vacuum properties so that it is advantageous to apply the heating support, in particular directly, to an enveloped vacuum mattress. Thus, already existing vacuum mattresses can also be retrofitted with a heating mat or a battery module of the initially mentioned type.

A liquid-repellent layer can additionally be arranged on the vacuum mattress. The heating support or further components of the vacuum mattress can hereby be protected against liquid. The liquid-repellent layer can be arranged on the heating support at the upper side of the vacuum mattress. It should be noted here that the upper side of the vacuum mattress is the side of the vacuum mattress at which a patient is treated.

An electrical cabling of the heating support can likewise be arranged at the upper side of the vacuum mattress and is preferably likewise arranged under the liquid-repellent layer so that the electrical cabling of the heating support can also be protected against liquids.

An electrical coupling of the electrical cabling can be provided and can be guided through the liquid-repellent layer to establish or to provide an electrical connection with the batten module.

The battery module can be arranged on the vacuum mattress such that the electrical coupling is covered by the battery module. In particular such that the electrical coupling is covered in a liquid-repellent manner by the battery module.

In accordance with a further aspect of the disclosure, the disclosure relates to a heating mat comprising a medical battery module, wherein the medical battery module is separate from the heating mat and is electrically connectable to a heating support of the heating mat.

The advantages that were discussed above in connection with the medical battery module apply accordingly to the vacuum mattress and to the heating mat.

The heating support can be arranged at an upper side of the heating mat. A liquid-repellent layer can likewise be arranged on the healing mat. The liquid-repellent layer can preferably be arranged on the heating support at the upper side of the heating mat.

An electrical cabling of the heating support can likewise be arranged at the upper side of the heating mat. A portion of the electrical cabling can in particular project from the heating mat. Here, the heating support of the heating mat can be particularly easily connected to the battery module remotely from the heating mat.

An electrical coupling of the electrical cabling can he provided that is arranged at the portion of the electrical cabling that is guided through the liquid-repellent layer and that partly projects from the heating mat to establish or to provide an electrical connection with the battery module. The battery module can be arranged or arrangeable remotely from or on the heating mat. The battery module can also have a cable having a negative feedback that can be coupled to the electrical coupling of the heating mat.

The advantages that were discussed above in connection with the medical battery module apply accordingly to the vacuum mattress and to the heating mat.

### BRIEF DESCRIPTION OF THE DRAWINGS

Embodiments of the disclosure will be explained in more detail with reference to the drawings.

FIG. 1 illustrates a view of a battery housing;

FIG. 2 illustrates a side view of a heating mat that is connected to a battery module in accordance with FIG. 1;

FIG. 3 illustrates a view of a vacuum mattress with a temperature-regulating film and electrical cabling;

FIG. 4 illustrates a view of the vacuum mattress of FIG. 3, wherein a liquid-repellent film is arranged on the temperature-regulating film;

FIG. 5 illustrates an exploded drawing of a further battery module;

FIG. 6 illustrates a view of a vacuum mattress with a fixedly installed battery module;

FIG. 7 illustrates a view of a further battery compartment or a battery housing;

FIG. 8 illustrates a side view of the battery housing of FIG. 7;

FIG. 9 illustrates a view of a battery that is arranged in a frame of the battery housing of FIG. 7;

FIG. 10 illustrates a view of the battery housing of FIG. 7 in which an upper shell has been removed;

FIG. 11 illustrates a view of the battery housing of FIG. 10 in which a board has been removed and the battery is not completely arranged in the battery compartment; and

FIG. 12 illustrates an exploded drawing of the components of FIG. 9.

### DETAILED DESCRIPTION

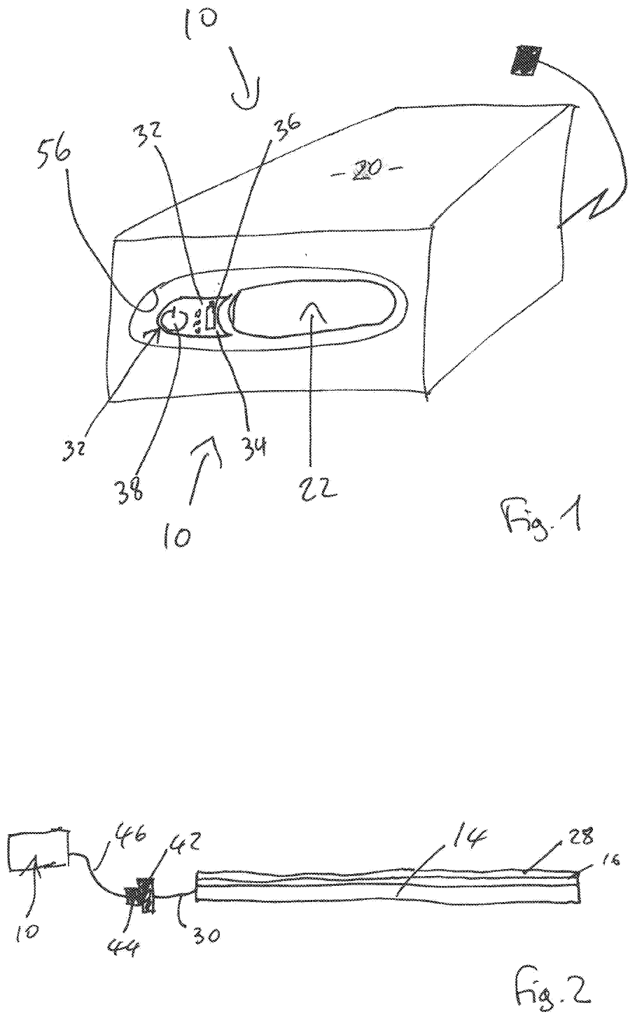

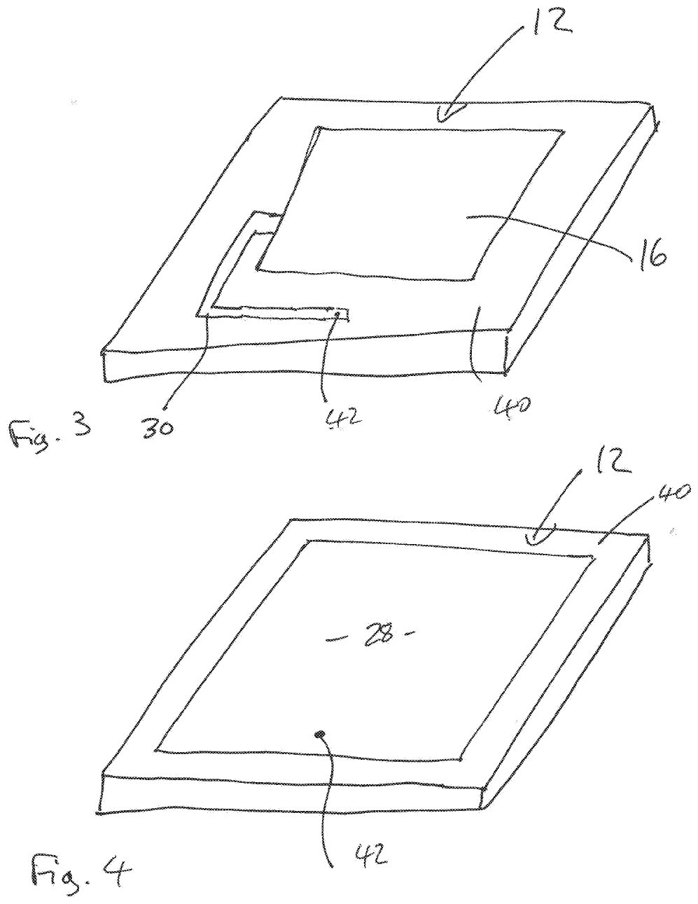

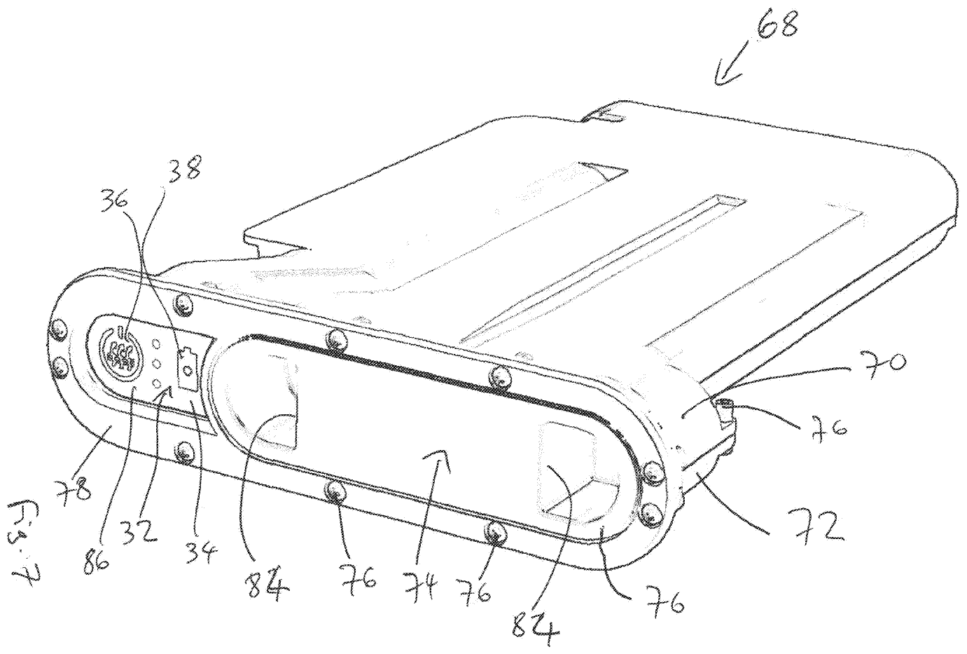

FIG. 1 shows a medical battery module 10 for vacuum mattresses 12 and heating mats 14, the medical battery module 10 comprising a battery compartment 56, a battery 22 that is removable from and insertable into the battery compartment 56, wherein the battery 22 is electrically connectable or connected to a heating support 16 (see also FIG. 2). The battery compartment 56 is padded with a shock-resistant material 18 and the shock-resistant material 18 is at least partly sheathed by a liquid-repellent film 20 (see also FIG. 5). The battery module 10 is either separate from the heating support or can lie on the heating support.

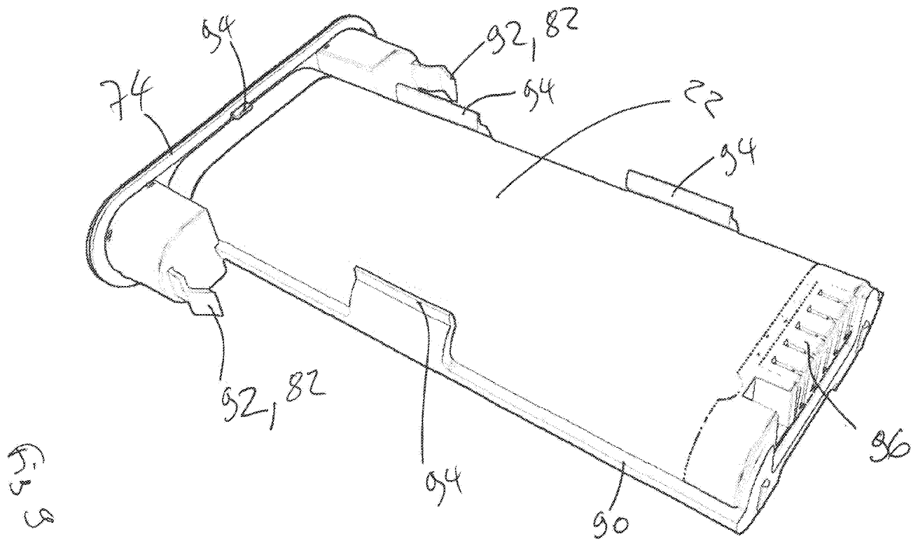

The battery compartment 56 is typically manufactured from the shock-resistant material 18 such that a battery housing 68 (see FIG. 7) from which the battery 22 can be removed or into which the battery 22 can be reinserted is arranged in the battery compartment 56. The battery housing 68 is manufactured from a plastic that is harder than the shock-resistant material 18 from which the battery compartment 56 is manufactured.

The thermoplastic can be a material from the group of thermoplastics comprising: acrylonitrile butadiene styrene (ABS), polyamides (PA), polylactide (PLA), polymethyl methacrylate (PMMA), polycarbonate (PC), polyethylene terephthalate (PET), polyethylene (PE), polypropylene (PP), polystyrene (PS), polyether ether ketone (PEEK), polyvinyl chloride (PVC), and combinations thereof.

The shock-resistant material 18 can comprise a material from the group comprising plastics, thermoplastics, foam comprising isocyanates and polyols, polyurethane, TPU, styrofoam, and the combinations thereof.

The battery module 10 comprises a control unit (electronic controller) by which a temperature of the heating support can be set. The control unit further comprises a control panel 34 having an on/off button 36 and a charge state display 38 for the battery 22.

FIG. 2 shows a partly sectioned representation of a heating mat 14 that is electrically connected to the battery module 10. In this respect, the medical battery module 10 is physically separate from the heating mat 14 and is electrically connected only to the heating support 16 of the heating mat 14.

For this purpose, a cable 46 of the battery module, which has a negative feedback 44, is coupled to the heating mat by an electrical coupling 42 of the heating mat 14. The electrical coupling 42 is electrically connected to the heating mat 16 via an electrical cabling 30.

Liquid-repellent couplings, which ensure an electrical coupling between the battery module and the heating mat and protect the electrical conductors of the latter against liquids, can be used as a negative feedback and an electrical coupling,

The heating support 16 is arranged at an upper side 34 of the heating mat 14. A liquid-repellent layer 28 is likewise arranged at the upper side 34 of the heating mat 14 on the heating support 16.

The electrical cabling 30 of the heating support 16 is likewise arranged at the upper side 34 of the heating mat 14, wherein a portion of the electrical cabling projects from the heating mat 14. At the portion of the liquid-repellent layer 28 at which the electrical cabling 30 is guided out of the heating mat, a seal (not shown) is provided to prevent an entry of liquid into the heating mat.

The electrical coupling 42 is arranged at the portion of the electrical cabling 30 that is guided through the liquid-repellent layer 28 and partly projects from the heating mat 14.

The liquid-repellent film 20 or the liquid-repellent layer 28 can comprise a material from the group of thermoplastic elastomers, such as thermoplastic polyurethane (TPU), foam, polyurethane, styrofoam, polystyrene, and further substances and their combinations thereof.

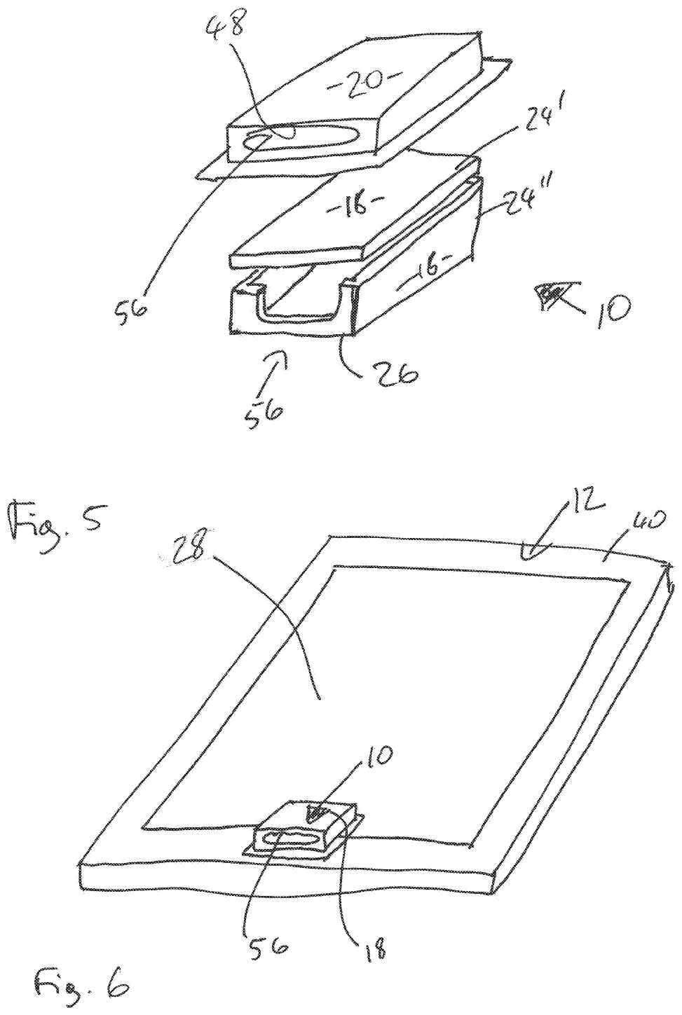

FIG. 3 shows a vacuum mattress 12, wherein the heating support 16 and the electrical cabling 30 of the heating support 16 are arranged at the upper side 34 of the vacuum mattress 12. The electrical cabling 30 is directly connected to the heating mat at one end and an electrical coupling 42, by which the heating support 16 is electrically connected to the battery module, is provided at its other end.

In FIG. 4, the liquid-repellent layer 28 is disposed both at the heating support 16 and at the upper side 40 of the vacuum mattress 12. The electrical cabling 30 is guided through the liquid-repellent layer 28 at the coupling 42 to establish or provide an electrical connection with the battery module 10 by the coupling 42.

The liquid-repellent layer 28 is dimensioned such that it covers both the electrical cabling 30 and the heating support 16; the shape of the heating support and of the arrangement of the electrical cabling 30 can deviate from the shape and arrangement shown, the only important thing here is that the heating support at least covers the body of a patient to heat or cool it and that the electrical cabling 30 and the heating support 16 can be covered by the liquid-repellent layer 28.

FIG. 5 shows that the battery compartment 56 comprises two half shells 24′, 24″that are each manufactured from the shock-resistant material 18. After an assembly of the battery compartment 56, it is completely sheathed by the liquid-repellent film 20 except for an opening at the front side of the battery compartment 56 and at its base 26. The battery housing 68 can be inserted into the battery module 10 either before the sheathing or before the assembly of the battery compartment 56 or it can be inserted into the battery compartment 56 after the sheathing of the battery compartment 56 by the liquid-repellent film 20.

When the battery module 10 is used with the heating mat 14, the battery compartment 56 is completely sheathed by the liquid-repellent film 20 except for the opening 48, i.e. also except for the base 26.

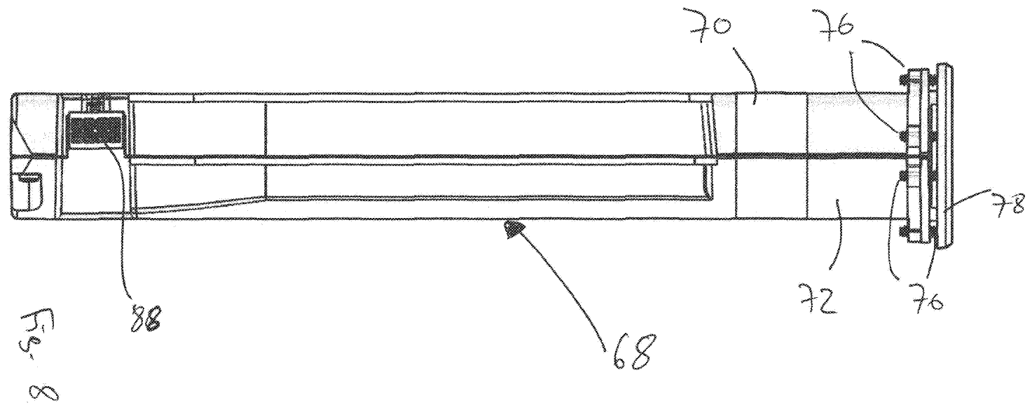

FIG. 6 shows the completed vacuum mattress 12 with the medical battery module 10. The base 26 of the medical battery module 10 is directly arranged on the vacuum mattress 12 such that the electrical coupling 42 is covered in a liquid-repellent manner by the battery module 10.

FIG. 7 shows a view of a battery housing 68. The battery housing 68 is fixedly installed in the compartment 58. The battery 22 can both be removed from and reinserted into the battery housing 68, for example, to be charged or maintained externally and separately from the vacuum mattress or the heating mat or a stretcher support.

The battery housing 68 is a multi-part housing that is at least liquid-repellent, preferably liquid-tight, to prevent an entry of liquid in the region of the battery in the vacuum mattress.

The battery housing 68 has an upper shell 70, a lower shell 72, and a cover 74, wherein the upper shell 70 is connected to the lower shell 72. Screw connections 76 are provided to improve the connection between the upper shell 70 and the lower shell 78. Furthermore, an outer frame 78 is connected to the upper shell 70 and to the lower shell 78 by screw connections 76. The screw connection typically comprises a bolt and a nut, but can also be formed by an integral internal thread or external thread that accordingly cooperate with a bolt or a nut.

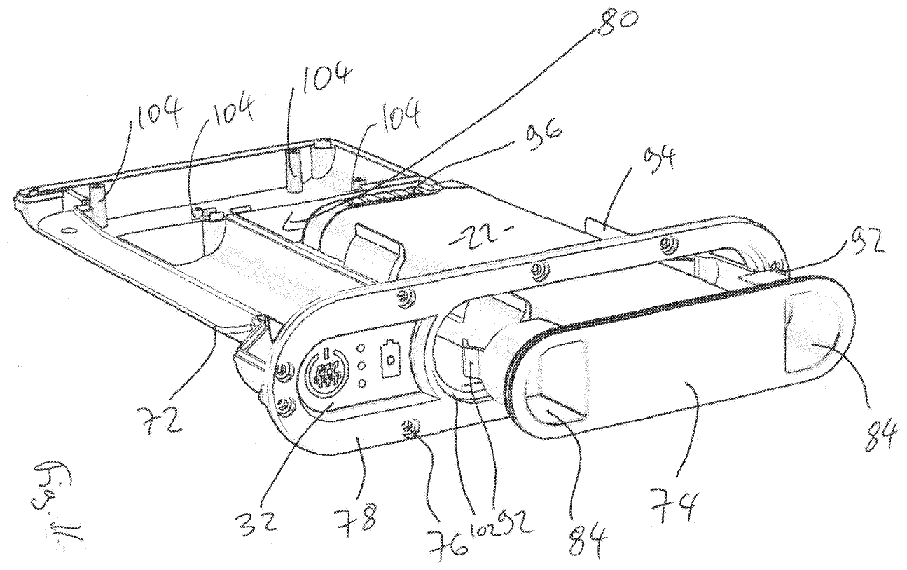

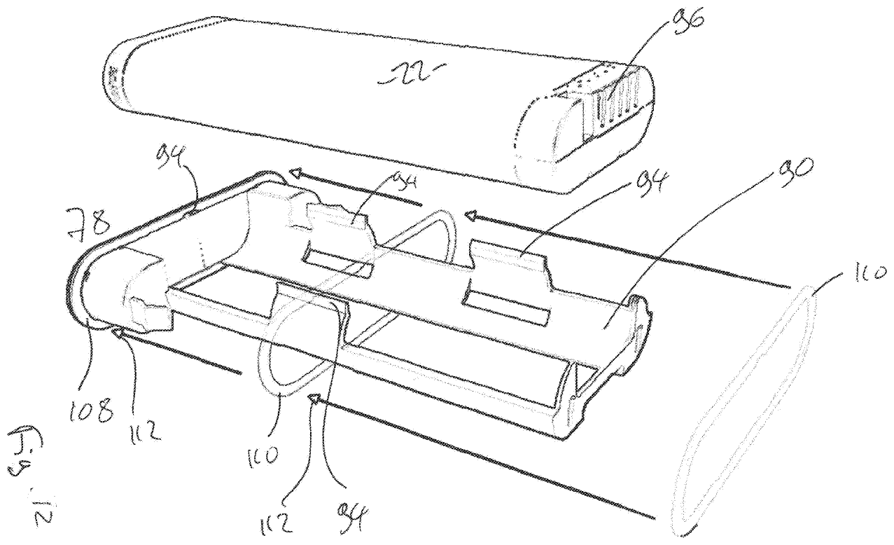

On the assembly, the cover 74 is inserted through the outer frame 78 into a reception space 80, which is formed between the upper shell 70 and the lower shell 72 (see FIGS. 10 and 11), and is releasably coupled to the upper shell 70 and to the lower shell 72.

The coupling between the frame and the upper shell 70 as well as the lower shell 72 is made possible by a releasable snap-in connection 82 (see FIG. 10). The snap-in connection 82 can be activated by a pressing in the cutouts 84 to release the connection, for example.

The control unit 32 is arranged at an outer visible side of the battery housing 68. This takes place in that a film circuit 86, on which the control panel 34 comprising the on/off button 36 and the charge state display 38 is arranged, is fastened to the upper shell 70, to the lower shell 72 and, if necessary, to the frame 78.

FIG. 8 shows a side view of the battery housing 68. An electrical interface 88 is provided at the rear end of the battery housing 68 that is disposed opposite the frame 78. The electronic components of the vacuum mattress, such as the heating mat 14, the regulation circuit 58, the microcontroller 60, the sensors 52, 54 and the medical devices 52′, 54′, can be supplied with power by this interface 88.

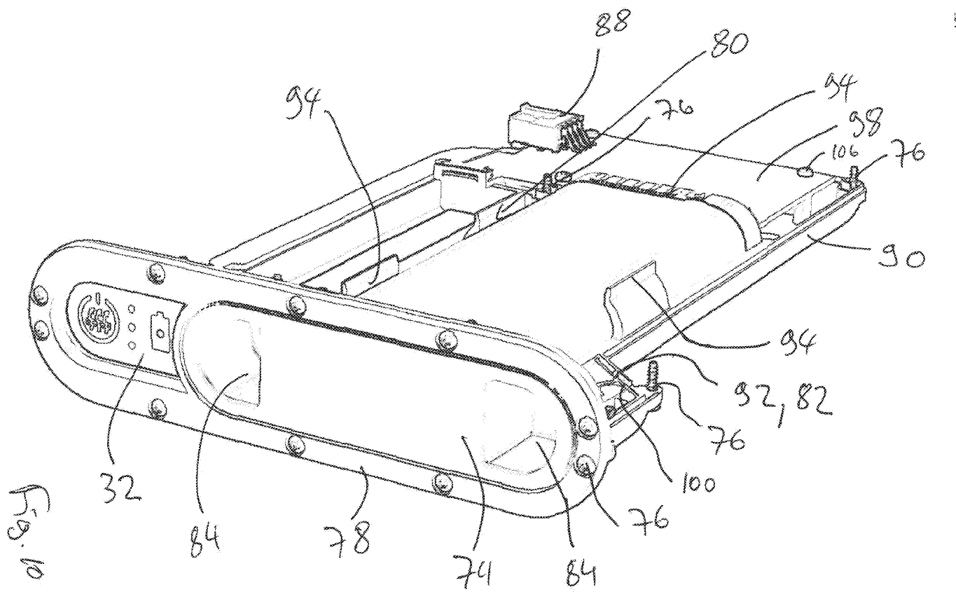

FIG. 9 shows a view of the battery 22 that is arranged in a frame 90 of the battery housing 68. The frame 90 is fastened to the cover 74 for this purpose. More precisely, the frame 90, together with the cover 74, is manufactured from plastic in an injection molding process.

To connect the cover to the upper shell 70 and the lower shell 72, snap hooks 92 are provided at one part of the cover 74 at whose oppositely disposed side the cutouts 84 are provided.

The battery 22 is connected in a form-fitting manner to both the cover 74 and the frame 90 by latch noses 94. The battery 22 can be removed from and reinserted into the battery housing 68 together with the cover 74 and the frame 90.

At its end disposed opposite the cover 74, the battery 22 has a battery interface 96 by which the battery 22 can be electronically connected to the interface 88.

FIG. 10 shows a view of the battery housing 68 in which the upper shell 70 has been removed. A board 98 is arranged in the rear end of the battery housing 68 and electronically connects the battery 22 via the battery interface 96 to the interface 88. The board is fastened to the lower shell 72 by screws 106, for example.

Furthermore, two cutouts 100 are disposed at the front end of the battery housing 68 and are configured to interact with the snap hooks 92. In FIG. 10, one of these cutouts 100 is covered by the outer frame 78 so that it is not visible.

FIG. 11 shows a view of the battery housing 68 in which the board 98 and the interface 88 have been removed and in which the battery 22 has been slightly pulled out of the battery housing 68. At the rear end of the battery housing 68, webs 104 are visible by which the board 98 can be fastened to the lower shell 72 by the screws 106.

The outer frame 78 has a groove 102 which the cover 74 contacts when the battery 22 is inserted.

FIG. 12 shows an exploded drawing in which the battery 22 is removed from the frame 90. The cover 74 has an inner side 108 that forms a planar surface. A seal 110 can likewise be seen that is arranged at this inner side 108 by pulling it onto the frame 90 in the direction of the arrow 112. This seal 110 enables a sealing between the cover 74, the upper shell 70, and the lower shell 72.

The upper shell 70, the lower shell 72, and the cover 74 are manufactured from a plastic, in particular a thermoplastic, preferably in an injection molding process. For example, the thermoplastic can be selected from the following list of thermoplastics: acrylonitrile butadiene styrene (ABS), polyamides (PA), polylactide (PEA), polymethyl methacrylate (PMMA), polycarbonate (PC), polyethylene terephthalate (PET), polyethylene (PE), polypropylene (PP), polystyrene (PS), polyether ether ketone (PEEK), and poly vinyl chloride (PVC),

The battery housing 68 is fixedly connected to the vacuum mattress and is arranged in the compartment 56. The snap-in connection 82 is provided to prevent the battery 22 from flying out of the battery housing. In addition to the snap-in connection 82, the battery 22 can also be held in the compartment 56 by the covering 62 such as is, for example, shown in FIG. 8.

1. A medical battery module for vacuum mattresses and heating mats, the medical battery module comprising:
a battery compartment; and
a battery that is removable from and insertable into the battery compartment, the battery electrically connectable to a heating support of the vacuum mattress or of the heating mat, the battery compartment being padded with a shock-resistant material and the shock-resistant material is at least partly sheathed by a liquid-repellent film, and the battery module is either separate from the heating support or lying on the heating support.

2. A medical battery module in accordance with claim 1, wherein the battery compartment is formed from the shock-resistant material and is configured to receive a battery housing formed from a plastic that is harder than the shock-resistant material.

3. A medical battery module in accordance with claim 1,
wherein the battery compartment comprises two half shells.

4. A medical battery module in accordance with claim 1,
wherein the shock-resistant material comprises two or more components.

5. A medical battery module in accordance with claim 1,
wherein a control unit of the battery and of the heating support is jointly arranged at one side of the medical battery module.

6. A medical battery module in accordance with claim 1,
wherein the shock-resistant material comprises a material selected from the group of plastics, thermoplastics, foam comprising isocyanates and polyols, polyurethane, TPU, styrofoam, and combinations thereof.

7. A medical battery module in accordance with claim 1,
wherein the liquid-repellent film comprises a material selected from the group of thermoplastic elastomers, such as thermoplastic polyurethane (TPU), foam, polyurethane, styrofoam, polystyrene, and further materials and combinations thereof.

8. A medical battery module in accordance with claim 2,
wherein the material of the battery housing comprises a plastic, in particular a thermoplastic.

9. A medical battery module in accordance with claim 8, wherein the plastic comprises a material from the group of thermoplastics comprising: acrylonitrile butadiene styrene (ABS), polyamides (PA). polylactide (PLA), poly methyl methacrylate (PMMA), polycarbonate (PC), polyethylene terephthalate (PET), polyethylene (PE), polypropylene (PP), polystyrene (PS), polyether ether ketone (PEEK), poly vinyl chloride (PVC), and combinations thereof.

10. A medical battery module in accordance with claim 1,
wherein a base of the medical battery module is disposed on the vacuum mattress or the heating mat.

11. A medical battery module in accordance with claim 1,
wherein the medical battery module is completely sheathed by the liquid-repellent film except for a front side of the battery compartment.

12. A medical battery module in accordance with claim 1,
wherein the battery compartment comprises a battery housing, and the battery is capable of being removed from the battery housing and inserted into the battery housing.

13. A medical battery module in accordance with claim 12, wherein
the battery housing comprises a multi-part housing that is at least liquid-repellent.

14. A medical battery module accordance with claim 1,
wherein the heating support has a temperature-regulating film configured to be regulated to an at least substantially constant temperature in a range from 15 to 42° C., the temperature capable of being set in dependence on a patient by a control unit.

15. A medical battery module in accordance with claim 12,
wherein the control unit is arranged at an outer visible side of the battery housing.

16. A medical battery unit in accordance with claim 12,
wherein the battery housing has an upper shell, a lower shell, and a cover, the upper shell is connected to the lower shell and the cover is releasably coupled to the upper shell and to the lower shell, by a releasable snap-in connection.

17. A medical battery module in accordance with claim 16, wherein the battery is connected to the cover, and the battery is capable of being removed from the battery housing and inserted into the battery housing together with the cover.

18. A medical battery module in accordance with claim 17, wherein the cover has a frame in which the battery is configured to be arranged.

19. A medical battery module in accordance with claim 16,
wherein a seal is arranged between the cover and the upper shell and the lower shell.

20. A medical battery module in accordance with claim 19, wherein the seal is arranged between the cover and a groove of an outer frame, and the outer frame connects the upper shell to the lower shell.

21. A medical battery module in accordance with claim 16,
wherein the battery housing or the cover is manufactured from a plastic.

22. A medical battery module in accordance with claim 19,
wherein the control unit is arranged in a region of the releasable snap-in connection between the upper shell and the lower shell.

23. A medical battery module accordance with claim 16,
wherein the battery housing has a reception space for at least one board on which at least one electronic circuit is provided to communicate with the battery.

24. A medical battery module in accordance with claim 1,
wherein the module or the battery is configured to output an acoustic signal or a visual signal as soon as a predefined charge state threshold has been undercut.

25. A vacuum mattress comprising a medical battery module in accordance with claim 1, wherein the medical battery module is welded or adhesively bonded to the vacuum mattress and is electrically connected to the heating support of the vacuum mattress.

26. A vacuum mattress in accordance with claim 25, wherein the heating support is arranged at an upper side of the vacuum mattress.

27. A vacuum mattress in accordance with claim 25,
wherein a liquid-repellent layer is arranged on the vacuum mattress.

28. A vacuum mattress in accordance with claim 26,
wherein the liquid-repellent layer is arranged on the heating support at the upper side of the vacuum mattress, and the liquid-repellent layer is manufactured from a same material as or a similar material to the liquid-repellent film.

29. A vacuum mattress in accordance with claim 26,
wherein an electrical cabling of the heating support is arranged at the upper side of the vacuum mattress.

30. A vacuum mattress in accordance with claim 29,
wherein an electrical coupling of the electrical cabling is provided and is guided through the liquid-repellent layer to provide an electrical connection with the battery module.

31. A vacuum mattress in accordance with claim 30,
wherein the battery module is arranged on the vacuum mattress such that the electrical coupling is covered by the battery module.

32. A vacuum mattress in accordance with claim 30,
wherein the battery module is arranged on the vacuum mattress such that the electrical coupling is covered in a liquid-repellent manner by the battery module.

33. A heating mat comprising:
a medical battery module in accordance with claim 1, the medical battery module being separate from the heating mat and electrically connectable to a heating support of the heating mat.

34. A heating mat in accordance with claim 33,
wherein the heating support is arranged at an upper side of the heating mat.

35. A heating mat in accordance with claim 33,
wherein a liquid-repellent layer is arranged on the heating mat.

36. A heating mat in accordance with claim 34,
wherein the liquid-repellent layer is arranged on the heating support at the upper side of the heating mat.

37. A heating mat in accordance with claim 34,
wherein an electrical cabling of the heating support is arranged at the upper side of the heating mat.

38. A heating mat in accordance with claim 37,
wherein a portion of the electrical cabling projects from the heating mat.

39. A heating mat in accordance with claim 38,
wherein an electrical coupling of the electrical cabling is arranged at a portion of the electrical cabling that is guided through the liquid-repellent layer and that partly projects from the heating mat to provide an electrical connection with the battery module.

40. A heating mat in accordance with claim 39,
wherein the battery module is arranged or can he configured to he arranged remotely from or on the heating mat.

41. A heating mat in accordance with claim 39, wherein the battery module has a cable comprising a negative feedback configured to be coupled to the electrical coupling of the heating mat.
